(19) United States
(12) Patent Application Publication
(10) Pub. No.: US 2016/0325206 A1
(43) Pub. Date: Nov. 10, 2016

(54) ACOUSTIC PRE-CONDITIONER

(71) Applicant: FloDesign Sonics, Inc., Wilbraham, MA (US)

(72) Inventors: Walter M. Presz, JR., Wilbraham, MA (US); Bart Lipkens, Hampden, MA (US); Jason Dionne, Simsbury, CT (US); Rudolf Gilmanshin, Framingham, MA (US); Erik Miller, Belchertown, MA (US)

(73) Assignee: FLODESIGN SONICS, INC.

(21) Appl. No.: 15/149,036

(22) Filed: May 6, 2016

Related U.S. Application Data
(60) Provisional application No. 62/157,492, filed on May 6, 2015.
(60) Provisional application No. 62/180,956, filed on Jun. 17, 2015.

(51) Int. Cl. B01D 21/28; C12M 1/00; B01D 21/00

(52) U.S. Cl. CPC B01D 21/283; B01D 21/0012; C12M 47/04

(57) ABSTRACT

Devices and methods for pre-conditioning and/or post-conditioning a host fluid containing a second fluid or particulate are disclosed. The devices include a flow chamber having first opening and a particulate outlet. The devices can also include side openings and alignment, fluid, and particulate screens. An ultrasonic transducer can be driven to create an acoustic standing wave in the flow chamber, or alternatively be driven to excite the wall of the flow chamber in which it is located. This creates a uniformly stratified flow within the flow chamber, with the second fluid or particulate being aligned in planes in the fluid mixture. This permits the host fluid to be separated therefrom using the fluid screen and the particulate screen.

### CROSS-REFERENCE TO RELATED APPLICATIONS

This application claims priority to U.S. Provisional Patent Application Ser. No. 62/157,492, filed on May 6, 2015; and to U.S. Provisional Patent Application Ser. No. 62/180,956, filed on Jun. 17, 2015, the disclosures of which are hereby fully incorporated by reference in their entireties.

### BACKGROUND

The separation of secondary fluids and particles from a host fluid in a primary fluid stream is a process requiring specialty filtration. There is a need for pre-conditioning and/or post-conditioning of the fluid stream that is infiltrated with the secondary fluid and/or particles so as to improve downstream separation and filtration.

### BRIEF DESCRIPTION

The present disclosure relates, in various embodiments, to acoustophoretic devices and methods of acoustically pre-conditioning and/or post-conditioning a host fluid to improve downstream processing and filtration of a secondary fluid or particulate in the host fluid. Briefly, an acoustic standing wave is used to align particles and/or a secondary fluid, separating them into discrete locations within the host fluid. The concentrated particles/secondary fluid can then be separated from the host fluid. This reduces the amount of particles that are present in the host fluid, improving downstream processing and filtration.

The devices described herein can acoustically pre-condition and/or post-condition a mixture of a host fluid and a secondary fluid or particulate by creating a stratified fluid flow, with alternating layers of clarified fluid and dispersed-species-rich fluid. These alternating and separated layers can then be channeled through one or more outlet fluid screen(s) or particulate screen(s) designed to separate the layers of clarified fluid from the layers of dispersed-species-rich fluid. This improves further downstream processes, such as acoustic separation. In particular embodiments, it is contemplated that the clarified fluid is further processed to separate other materials from the fluid. In other embodiments, it is contemplated that the particles are subsequently purified and collected.

Disclosed herein are acoustophoretic devices comprising: a flow chamber having a particulate outlet at a first end of the flow chamber and a first opening at a second end of the flow chamber opposite the first end thereof; at least one ultrasonic transducer located on a wall of the flow chamber, the at least one ultrasonic transducer including a piezoelectric material driven by a voltage signal to create an acoustic standing wave in the flow chamber; a reflector located on a wall on the opposite side of the flow chamber from the at least one ultrasonic transducer; at least one opening located on a wall of the flow chamber between the reflector and the at least one ultrasonic transducer; an optional particulate screen located between the first opening and the flow chamber, the particulate screen including a plurality of slots therein; and a fluid screen located between the particulate outlet and the flow chamber, the fluid screen including a plurality of slots therein.

In certain embodiments, the slots in the particulate screen and the slots in the fluid screen have a width equal to about one-quarter of the wavelength of the acoustic standing wave. The slots in the particulate screen and the slots in the fluid screen can have a width of between about 0.005 inches and 0.02 inches and a length of between about 0.25 inches and 0.75 inches. For example, when the acoustic standing wave is operating at a frequency of 2.24 MHz, a quarter wavelength is on the order of 179 micrometers (μm), or 0.0066 inches. Thus, the width of the slots is matched to the wavelength of the acoustic standing wave. The height is sized to the cross-sectional area needed to accommodate the clarified fluid or particulate flow going through the slots.

In particular constructions, the acoustophoretic device can include at least one alignment screen located between the at least one side opening and the flow chamber, the alignment screen including a plurality of slots therein. The slots in the alignment screen can be sized as appropriate. Alignment screens are particularly contemplated for use when the mixture of host fluid and particles flows into the flow chamber through the side opening(s).

The acoustic standing wave can be a multi-dimensional acoustic standing wave. In other embodiments, the acoustic standing wave can be a planar acoustic standing wave. Further yet, in particular embodiments, the acoustic standing wave may be a combination of a planar acoustic standing wave and a multidimensional acoustic standing wave, where the planar acoustic standing wave and multidimensional acoustic standing wave are super-positioned on each other.

The methods described herein can acoustically pre-condition a mixture of a host fluid and a secondary fluid or particulate by cavitation prior to aligning the secondary fluid or particulate into planes. The cavitation would create micro-bubbles that assist in flocculation or aggregation of the particles during downstream processing.

In accordance with the present disclosure, methods are disclosed for pre-conditioning and/or post-conditioning a second fluid or a particulate within a host fluid, comprising: flowing a mixture of the host fluid and the second fluid or particulate through an acoustophoretic device and sending a voltage signal to drive the at least one ultrasonic transducer to create the acoustic standing wave in the flow chamber to create a uniformly stratified flow therein, such that the second fluid or particulate is aligned in planes in the mixture. The acoustophoretic device of the method comprises: a flow chamber having a particulate outlet at a first end of the flow chamber and a first opening at a second end of the flow chamber opposite the first end thereof; at least one ultrasonic transducer located on a wall of the flow chamber, the at least one ultrasonic transducer including a piezoelectric material driven by a voltage signal to create an acoustic standing wave in the flow chamber; a reflector located on a wall on the opposite side of the flow chamber from the at least one ultrasonic transducer; at least one side opening located on a wall of the flow chamber between the reflector and the at least one ultrasonic transducer; an optional particulate screen located between the first opening and the flow chamber, the particulate screen including a plurality of slots therein; and a fluid screen located between the particulate outlet and the flow chamber, the fluid screen including a plurality of slots therein

The slots in the particulate screen and the slots in the fluid screen may have a width equal to about one-quarter of the wavelength of the multi-dimensional standing wave. The slots in the particulate screen and the slots in the fluid screen can have a width of between about 0.005 inches and 0.02 inches and a height of between about 0.25 inches and 0.75 inches. In particular embodiments, the slots in the particulate screen and the slots in the fluid screen are arranged in two rows of longitudinal slots separated by a divider running therebetween.

The acoustophoretic device may further comprise at least one alignment screen located between the at least one side opening and the flow chamber, the alignment screen including a plurality of slots therein. The slots in the alignment screen can have a width of between about 0.005 inches and 0.02 inches and a height of between about 0.25 inches and about 0.75 inches.

The acoustic standing wave can be a multi-dimensional acoustic standing wave. In other embodiments, the acoustic standing wave can be a planar acoustic standing wave. Further yet, in particular embodiments, the acoustic standing wave may be a combination of a planar acoustic standing wave and a multidimensional acoustic standing wave, where the planar acoustic standing wave and multidimensional acoustic standing wave are super positioned on each other.

In particular embodiments, the at least one transducer and the reflector define a primary transducer-reflector pair, and the acoustophoresis device further comprises a secondary transducer-reflector pair located upstream of the primary transducer-reflector pair, the secondary transducer-reflector causing cavitation resulting in micro-bubbles in the host fluid that assist in flocculation or aggregation of the second fluid or particulate by the primary transducer-reflector pair.

In certain constructions, the slots in the particulate screen are aligned with the acoustic standing wave so as to permit the passage of the host fluid that has been clarified by the acoustic standing wave therethrough, while retarding the passage of the second fluid or particulate that has been concentrated by the acoustic standing wave therethrough; and the slots in the fluid screen are aligned with the acoustic standing wave so as to permit the passage of the second fluid or particulate that has been concentrated by the acoustic standing wave therethrough, while retarding the passage of the host fluid that has been clarified by the acoustic standing wave therethrough.

The methods described herein can acoustically pre-condition a mixture of a host fluid and a secondary fluid or particulate by setting an entire acoustophoretic device into vibration to create a uniformly stratified flow, with alternating layers of clarified and dispersed-species-rich fluid. The wall of the acoustophoretic device in which a transducer is located can be excited at the wall's resonant frequency to cause standing waves inside the device to separate particles in a host fluid flowing therethrough to align into planes.

In accordance with the present disclosure, methods are disclosed for pre-conditioning and/or post-conditioning a second fluid or a particulate within a host fluid, the method comprising: flowing a mixture of the host fluid and the second fluid or particulate through an acoustophoretic device and sending a voltage signal to drive the at least one ultrasonic transducer to excite the wall of the flow chamber and create the acoustic standing wave in the flow chamber to create a uniformly stratified flow therein, such that the second fluid or particulate is aligned in planes in the flow chamber. The acoustophoretic device of the method comprises: a flow chamber having a particulate outlet at a first end of the flow chamber and a first opening at a second end of the flow chamber opposite the first end thereof; at least one ultrasonic transducer located upon a wall of the flow chamber, the at least one ultrasonic transducer including a piezoelectric material driven by a voltage signal to excite the wall of the flow chamber and create an acoustic standing wave in the flow chamber; and a reflector located on the opposite side of the flow chamber from the at least one ultrasonic transducer.

In certain embodiments, the acoustophoretic device further comprises at least one side opening located on a wall of the flow chamber between the reflector and the at least one ultrasonic transducer.

At least one particulate screen may be located between the at least one side opening and the flow chamber, the at least one particulate screen including a plurality of slots therein that are aligned with the acoustic standing wave so as to permit the passage of the host fluid that has been clarified by the acoustic standing wave therethrough, while retarding the passage of the second fluid or particulate that has been concentrated by the acoustic standing wave therethrough.

A fluid screen may be located between the particulate outlet and the flow chamber, the fluid screen including a plurality of slots therein that are aligned with the acoustic standing wave so as to permit the passage of the second fluid or particulate that has been concentrated by the acoustic standing wave therethrough, while retarding the passage of the host fluid that has been clarified by the acoustic standing wave therethrough.

In particular embodiments, the acoustic standing wave may be a multi-dimensional acoustic standing wave. Examples of such multi-dimensional acoustic standing waves can be found in commonly owned U.S. Pat. No. 9,228,183, the entire contents of which are hereby fully incorporated by reference.

These and other non-limiting characteristics are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 illustrates an exploded view of a first exemplary acoustophoretic device according to the present disclosure including alignment, particulate, and fluid screens for pre-conditioning and/or post-conditioning a fluid flowing therethrough.

FIG. 2 is a cross-sectional view of the acoustophoretic device of FIG. 1, illustrating the flow of fluid through the device from the side openings to the first opening and the removal of a second fluid or particulate therefrom via the particulate outlet.

FIG. 3 illustrates an exemplary fluid screen having slots therein according to the present disclosure.

FIG. 4 illustrates an exemplary particulate screen having slots therein according to the present disclosure.

FIG. 5 illustrates a cross-sectional view of the acoustophoretic device of FIG. 1, showing the alignment of the slots in the fluid and particulate screens with the planes of concentrated particles and streams of clarified fluid between the particle planes in the flow chamber.

FIG. 6 illustrates an exemplary alignment screen having slots therein according to the present disclosure.

FIG. 7 is a cross-sectional view of a second exemplary acoustophoretic device according to the present disclosure, illustrating the flow of fluid through the device from the first opening at the top of the device to the side openings, and the removal of a second fluid or particulate therefrom via the particulate outlet at the bottom of the device.

FIG. 8 is a cross-sectional view of a third exemplary acoustophoretic device according to the present disclosure, illustrating the flow of fluid through the device from the first opening at the bottom of the device to the side openings, and the removal of a second fluid or particulate therefrom via the particulate outlet at the top of the device.

FIG. 9 illustrates an exemplary setup of an ultrasonic transducer and reflector, arranged on opposite walls of a flow chamber and configured to pre-condition a fluid to create a uniformly stratified flow, with alternating layers of clarified fluid and dispersed-species-rich fluid to align material entrained in the fluid into planes.

FIG. 10 illustrates an exemplary setup of a primary transducer-reflector pair and a secondary transducer-reflector pair, the secondary transducer-reflector pair configured to cause cavitation in the fluid to create micro-bubbles therein, thereby assisting in aggregation or flocculation of material in the fluid by the first transducer-reflector pair.

FIG. 11 illustrates an exemplary setup of ultrasonic transducer(s) arranged on the exterior side of the wall(s) of a flow chamber The ultrasonic transducer(s) is configured to excite the wall of the device to create a standing wave in the flow chamber and separate particles in a fluid therein into planes, thereby creating a uniformly stratified fluid flow, with alternating layers of clarified and dispersed-species-rich fluid.

FIG. 12 is a cross-sectional diagram of a conventional ultrasonic transducer.

FIG. 13 is a cross-sectional diagram of an ultrasonic transducer according to the present disclosure. An air gap is present within the transducer, and no backing layer or wear plate is present.

FIG. 14 is a cross-sectional diagram of an ultrasonic transducer according to the present disclosure. An air gap is present within the transducer, and a backing layer and wear plate are present.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named component and allowing the presence of other components. The term “comprising” should be construed to include the term “consisting of”, which allows the presence of only the named component, along with any impurities that might result from the manufacture of the named component.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of “from about 2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

It should be noted that many of the terms used herein are relative terms. For example, the terms “upper” and “lower” are relative to each other in location, i.e. an upper component is located at a higher elevation than a lower component in a given orientation, but these terms can change if the device is flipped. The terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, i.e. the flow fluids through an upstream component prior to flowing through the downstream component. It should be noted that in a loop, a first component can be described as being both upstream of and downstream of a second component.

The terms “horizontal” and “vertical” are used to indicate direction relative to an absolute reference, i.e. ground level. However, these terms should not be construed to require structures to be absolutely parallel or absolutely perpendicular to each other. For example, a first vertical structure and a second vertical structure are not necessarily parallel to each other. The terms “top” and “bottom” or “base” are used to refer to surfaces where the top is always higher than the bottom/base relative to an absolute reference, i.e. the surface of the earth. The terms “upwards” and “downwards” are also relative to an absolute reference; upwards is always against the gravity of the earth.

The term “parallel” should be construed in its lay sense of two surfaces that maintain a generally constant distance between them, and not in the strict mathematical sense that such surfaces will never intersect when extended to infinity.

The present application refers to “the same order of magnitude.” Two numbers are of the same order of magnitude if the quotient of the larger number divided by the smaller number is a value of at least 1 and less than 10.

Acoustophoresis is the separation of particles and secondary fluids from a primary or host fluid using high intensity acoustic standing waves, and without the use of membranes or physical size exclusion filters. It has been known that high intensity standing waves of sound can exert forces on particles in a fluid when there is a differential in both density and/or compressibility, otherwise known as the acoustic contrast factor. The pressure profile in a standing wave contains areas of local minimum pressure amplitudes at its nodes and local maxima at its anti-nodes. Depending on the density and compressibility of the particles, they will be trapped at the nodes or anti-nodes of the standing wave. Generally, the higher the frequency of the standing wave, the smaller the particles that can be trapped due the pressure of the standing wave.

When acoustic standing waves propagate in liquids, the fast oscillations may generate a non-oscillating force on particles suspended in the liquid or on an interface between liquids. This force is known as the acoustic radiation force. The force originates from the non-linearity of the propagating wave. As a result of the non-linearity, the wave is distorted as it propagates and the time-averages are nonzero. By serial expansion (according to perturbation theory), the first non-zero term will be the second-order term, which accounts for the acoustic radiation force. The acoustic radiation force on a particle, or a cell, in a fluid suspension is a function of the difference in radiation pressure on either side of the particle or cell. The physical description of the radiation force is a superposition of the incident wave and a scattered wave, in addition to the effect of the non-rigid particle oscillating with a different speed compared to the surrounding medium thereby radiating a wave. The following equation presents an analytical expression for the acoustic radiation force on a particle, or cell, in a fluid suspension in a planar standing wave.

$${F}_{R} = \frac{3 π {P}_{0}^{2} {V}_{P} {β}_{m}}{2 λ} ϕ ( β , ρ ) sin ( 2 kx ) \qquad ( 1 )$$

where βₘ is the compressibility of the fluid medium, ρ is density, φ is acoustic contrast factor, Vₚ is particle volume, λ is wavelength, k is 2π/λ, P₀ is acoustic pressure amplitude, x is the axial distance along the standing wave (i.e., perpendicular to the wave front), and

$$ϕ ( β , ρ ) = \frac{5 {ρ}_{ρ} - 2 {ρ}_{m}}{2 {ρ}_{ρ} + {ρ}_{m}} - \frac{{β}_{ρ}}{{β}_{m}}$$

where ρₚ is the particle density, ρₘ is the fluid medium density, βₚ is the compressibility of the particle, and βₘ is the compressibility of the fluid medium.

For a multi-dimensional standing wave, the acoustic radiation force is a three-dimensional force field, and one method to calculate the force is Gor'kov's method, where the primary acoustic radiation force F_{R} is defined as a function of a field potential U, F_{V}=−∇(U), where the field potential U is defined as

$$U = {V}_{0} [ \frac{〈 {p}^{2} ( x , y , t ) 〉}{2 {ρ}_{f} {c}_{f}^{2}} {f}_{1} - \frac{3 {ρ}_{f} 〈 {v}^{2} ( x , y , t ) 〉}{4} {f}_{2} ]$$

and f₁ and f₂ are the monopole and dipole contributions defined by

$${f}_{1} = 1 - \frac{1}{{Λσ}^{2}} {f}_{2} = \frac{2 ( Λ - 1 )}{2 Λ + 1} , \\ where$$

$$σ = \frac{{c}_{ρ}}{{c}_{f}} Λ = \frac{{ρ}_{ρ}}{{ρ}_{f}} {β}_{f} = \frac{1}{{ρ}_{f} {c}_{f}^{2}}$$

where p is the acoustic pressure, u is the fluid particle velocity, Λ is the ratio of cell density ρₚ to fluid density ρ_{f}, σ is the ratio of cell sound speed cₚ to fluid sound speed c_{f}, Vₒ is the volume of the cell, and < > indicates time averaging over the period of the wave.

Gork'ov's model is for a single particle in a standing wave and is limited to particle sizes that are small with respect to the wavelength of the sound fields in the fluid and the particle. It also does not take into account the effect of viscosity of the fluid and the particle on the radiation force. As a result, this model cannot be used for the macro-scale ultrasonic separators discussed herein since particle clusters can grow quite large. A more complex and complete model for acoustic radiation forces that is not limited by particle size was therefore used. The models that were implemented are based on the theoretical work of Yurii Ilinskii and Evgenia Zabolotskaya as described in AIP Conference Proceedings, Vol. 1474-1, pp. 255-258 (2012). These models also include the effect of fluid and particle viscosity, and therefore are a more accurate calculation of the acoustic radiation force.

The present disclosure relates to acoustophoretic devices and methods that employ multi-dimensional ultrasonic acoustic standing waves, planar acoustic standing waves or combinations of planar and multidimensional acoustic standing waves (collectively referred to herein simple as acoustic standing waves) to acoustically precondition a host fluid to improve downstream processing and filtration of a secondary fluid or particulate in the host fluid. The acoustophoretic devices and methods disclosed herein use the axial radiation forces of a multi-dimensional acoustic standing wave. The axial radiation forces in a standing wave can be significantly higher than the lateral forces, though they are within an order of magnitude. Thus, significant performance improvements can be generated by using axial, rather than lateral, radiation forces to precondition particles or cells in a fluid suspension. For purposes of this disclosure, a standing wave where the lateral force is not the same order of magnitude as the axial force is considered a “planar acoustic standing wave.” Briefly, the acoustic standing waves cause particles within a controlled size range to be pushed into planes within the host fluid. This results in layers of concentrated particles and layers of clarified host fluid. The particles can then be passed through slots in an outlet screen, and the clarified host fluid exits through other outlets.

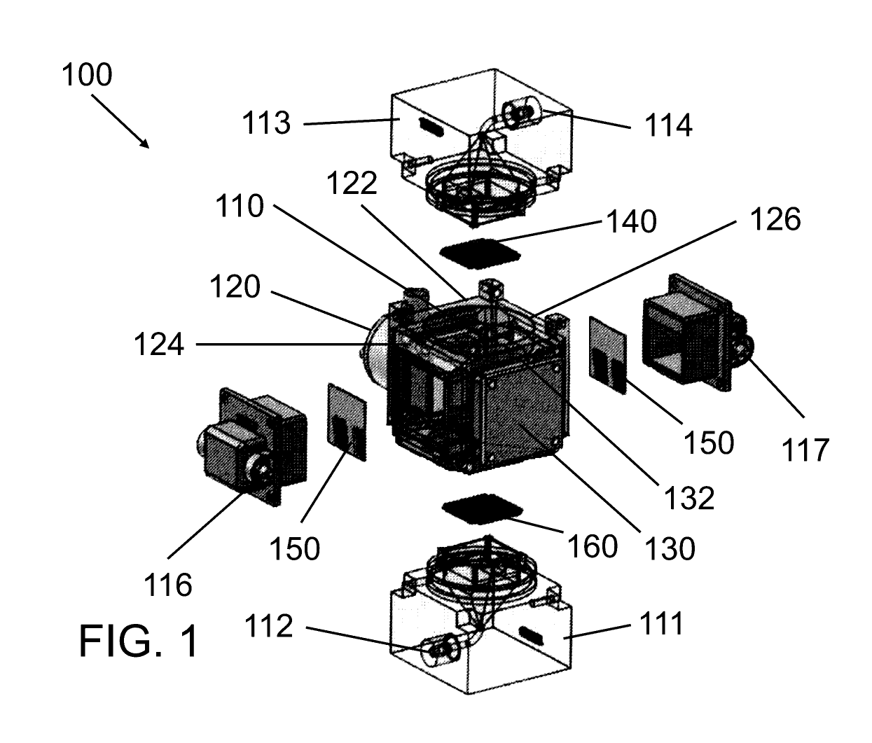

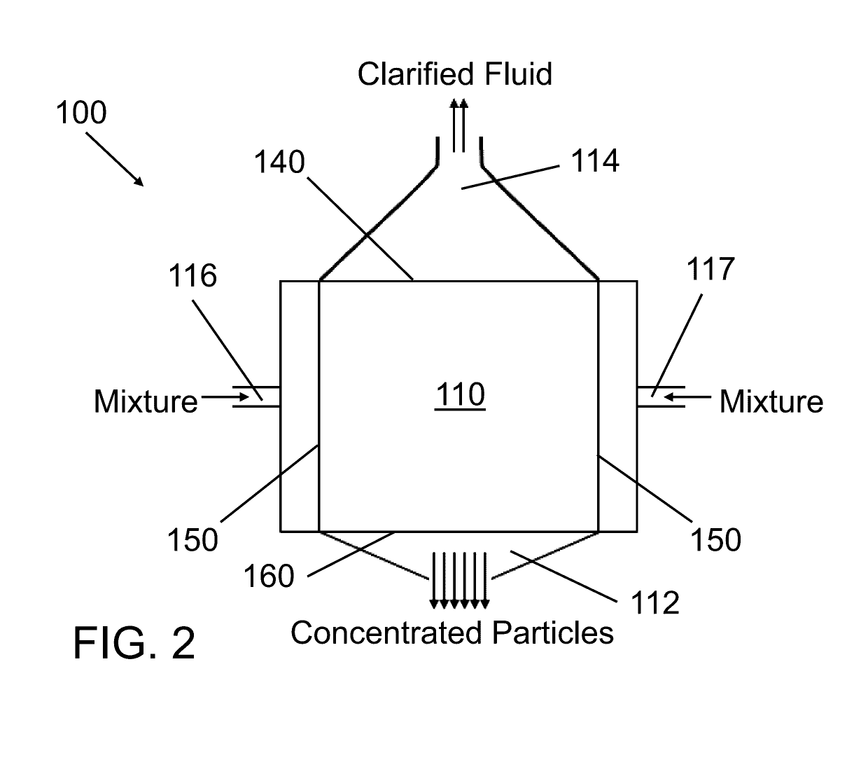

FIG. 1 presents an exploded view of a first exemplary embodiment of such an axial force acoustophoretic device designated generally as 100. FIG. 2 provides a cross-sectional view of the acoustophoretic device 100 depicted in FIG. 1, further illustrating various features and components thereof, and illustrating a particular operating method. The acoustophoretic device 100 generally operates so as to use the axial radiation forces from an acoustic standing wave. The acoustophoretic device 100 depicted in FIG. 1 includes a flow chamber 110, an ultrasonic transducer 120, a reflector 130, an optional particulate screen 140, and a fluid screen 160.

The flow chamber 110 is the region of the device 100 through which is flowed an initial mixture of the host fluid and a second fluid or particulate. In the embodiment shown in FIG. 1, the flow chamber 110 is defined by walls 122, 132, 124, and 126. More particularly, wall 122 serves as the wall on which the ultrasonic transducer 120 is located and wall 132 serves as the wall on which the reflector 130 is located. In the embodiment shown in FIG. 1, wall 122 is located opposite wall 132 (i.e., on an opposite side of the flow chamber 110), such that the ultrasonic transducer 120 and reflector 130 are located opposite one another.

In the embodiment of the device 100 depicted in FIG. 1, the flow chamber 110 includes a first opening 114, a particulate outlet 112, and at least one side opening 116, 117. The side openings 116 and 117 are located on walls of the flow chamber 110 between the reflector 130 and the transducer 120. For example, in the embodiment of FIG. 1, side opening 116 is located on wall 124, and side opening 117 is provided on wall 126. In this way, side opening 116 is located opposite side opening 117. The side openings 116 and 117 are located within the flow chamber at generally the same level as the transducer 120 and the reflector 130. Put another way, the side openings 116 and 117, the transducer 120, and the reflector 130 are equidistant from the first opening 114, or are equidistant from the particulate outlet 112.

The particulate outlet 112 is located at a first end 111 of the flow chamber 110 and generally allows for egress or collection of particles, cells, or the like from the flow chamber 110. The first opening 114 is located at a second end 113 of the flow chamber 110 and generally allows for egress of the residual fluid from the flow chamber 110. In the embodiment depicted in FIG. 1, the first end 111 is opposite the second end 113.

When the mixture of host fluid and particles passes through the acoustic standing waves, the mixture is separated into two different types of layers. One layer type has a higher concentration of particles relative to the incoming mixture, and one layer type has a lower concentration of particles relative to the incoming mixture (i.e. a layer of clarified fluid). These types of layers alternate within the flow chamber, so that planes of particles are located between the clarified fluid layers.

The device 100 can further includes a particulate screen 140 located between the first opening 114 and the flow chamber 110. The particulate screen 140 is designed to separate the particle streams (i.e., planes with particles) and the clarified fluid streams (i.e., the space between the planes of particles) from each other. As will be explained in greater detail herein, in the operating method illustrated depicted in FIG. 2, the particulate screen is particularly designed to allow the passage of clarified fluid therethrough, while preventing or retarding the passage of particles or particulate matter therethrough. As seen in FIG. 1, the particulate screen 140 is relatively flat. It is specifically noted that the particulate screen is optional, and does not need to be present for all operating methods.

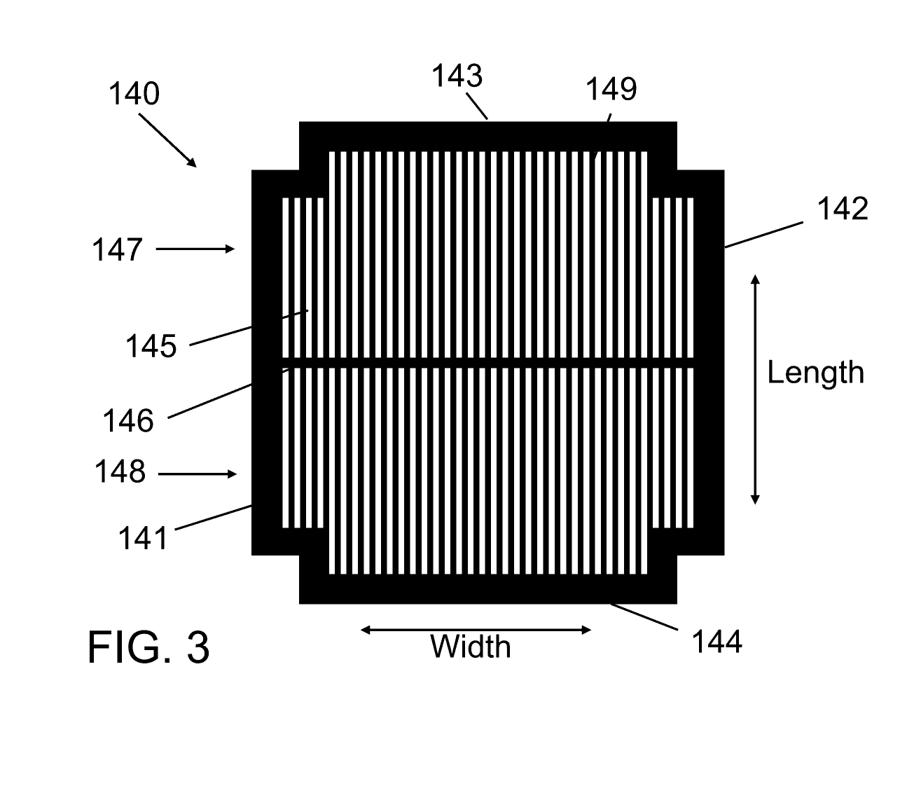

A plan view of the particulate screen 140 is shown in FIG. 3. The particulate screen has a first side 141, a second side 142 opposite the first side, a third side 143 between the first side and the second side, and a fourth side 144 opposite the third side and also located between the first side and the second side. The first side 141 and the second side 142 define a width, and the third side 143 and the fourth side 144 define a length, of the particulate screen. These four sides define a perimeter of the particulate screen. The exact shape of the perimeter is not significant, other than to ensure that clarified fluid is capable of passing through the slots 145, while the previously aligned and separated particles/particulate matter is retained in the flow chamber 110 by the particulate screen.

The particulate screen 140 includes a plurality of slots 145 therein, and a plurality of bars 149. Each slot is surrounded by two bars, and each bar is surrounded by two slots. As shown in FIG. 3, the slots 145 are longitudinal slots running both the length and width of the particulate screen 140. Put another way, the slots 145 in the particulate screen 140 run lengthwise from the top to the bottom thereof (i.e., from the third side 143 to the fourth side 144) and also span the width of the particulate screen 140 from one side to another (i.e., from the first side 141 to the second side 142). In particular embodiments, the slots 145 in the particulate screen 140 are arranged in two rows 147, 148, with a divider 146 running therebetween. The divider 146 may be necessary when fluid flowed through the device is flowed at high flow rates, to enhance the structural integrity to the particulate screen 140.

In the embodiment shown in FIG. 1, the slots 145 in the particulate screen 140 generally permit the clarified fluid flowing between the planes of particles to pass therethrough, while the previously aligned and highly concentrated particle planes are held back by the bars. The general result is that the clarified fluid passes through the particulate screen 140 toward the second end 113 of the device and subsequently out of the device via the first opening 114 of the device.

In certain embodiments, the slots 145 in the particulate screen 140 have a width equal to about one-quarter of the wavelength of the acoustic standing wave generated in the flow chamber 110 of the device. In other embodiments, the slots 145 in the particulate screen 140 can have a width of between about 0.005 inches and 0.02 inches and a length of between about 0.25 inches and 0.75 inches. Again, the slots 145 in the particulate screen 140 are aligned with the clarified fluid flowing between the planes of particles in the flow chamber 110. The width of the slots is appropriately matched to the frequency of the acoustic standing wave so as to maximize the passage of clarified fluid through the particulate screen 140 and out the first opening 114 of the device.

Referring back now to FIG. 1, the device 100 further includes a fluid screen 160 located between the particulate outlet 112 and the flow chamber 110. Similar to the particulate screen 140, the fluid screen 160 is likewise designed to separate the previously aligned particle streams (i.e., planes with particles) and the clarified fluid streams (i.e., the space between the planes of particles) from each other. As will be explained in greater detail herein, in the embodiment depicted in FIG. 1, the fluid screen is particularly designed to allow the passage of particles or particulate matter therethrough, while preventing or retarding the passage of clarified fluid therethrough. Put another way, the fluid screen operates in the opposite way of the particulate screen. As seen in FIG. 1, the fluid screen 160 is relatively flat.

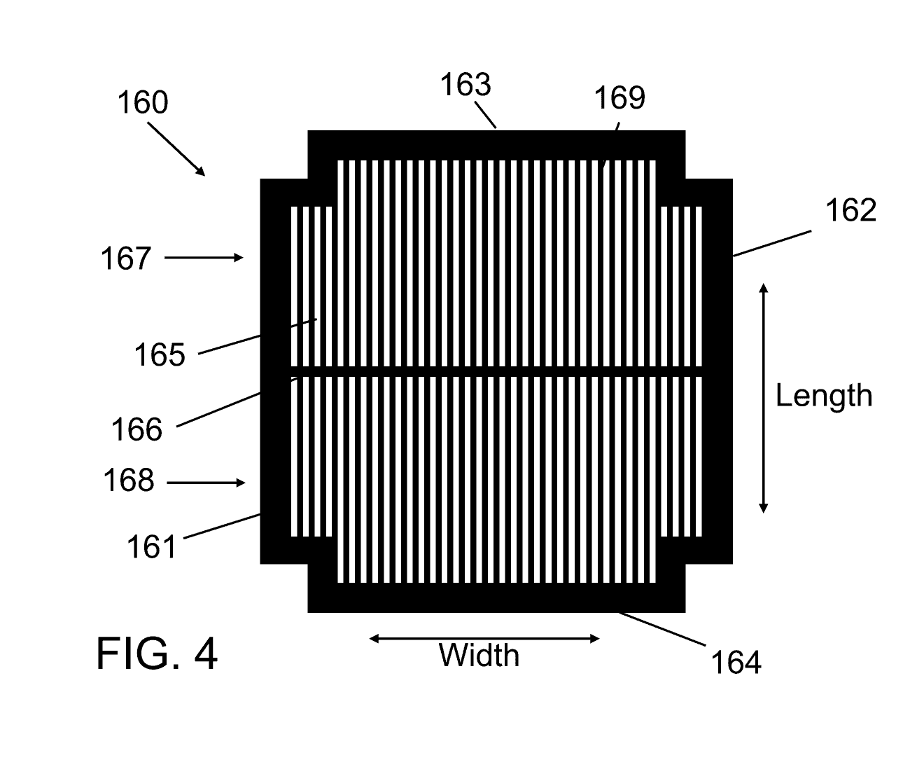

A plan view of the fluid screen 160 is shown in FIG. 4. The particulate screen has a first side 161, a second side 162 opposite the first side, a third side 163 between the first side and the second side, and a fourth side 164 opposite the third side and also located between the first side and the second side. The first side 161 and the second side 162 define a width, and the third side 163 and the fourth side 164 define a length, of the fluid screen. These four sides define a perimeter of the fluid screen. The exact shape of the perimeter is not significant, other than to ensure that the particles/particulate matter previously aligned and separated by the acoustic standing waves are capable of passing through the slots 145, while clarified fluid is prevented or retarded by the fluid screen from exiting the flow chamber 110 at the first end 111 of the device.

The fluid screen 160 includes a plurality of slots 165 therein, and a plurality of bars 169. Each slot is surrounded by two bars, and each bar is surrounded by two slots. As shown in FIG. 4, the slots 165 are longitudinal slots running both the length and width of the fluid screen 160. Put another way, the slots 165 in the particulate screen 160 run lengthwise from the top to the bottom thereof (i.e., from the third side 163 to the fourth side 164) and also span the width of the fluid screen 160 from one side to another (i.e., from the first side 161 to the second side 162). In particular embodiments, the slots 165 in the fluid screen 160 are arranged in two rows 167, 168, with a divider 166 running therebetween. The divider 166 may be necessary when fluid flowed through the device is flowed at high flow rates, to enhance the structural integrity to the fluid screen 160.

In the embodiment shown in FIG. 1, the slots 165 in the fluid screen 160 generally permit the highly concentrated particle planes to pass therethrough, while the clarified fluid flowing between the planes of particles are held back by the bars. The general result is that the highly concentrated particle planes pass through the fluid screen 160 toward the first end 111 of the device and subsequently out of the device via the particulate outlet 112 of the device.

In certain embodiments, the slots 165 in the fluid screen 160 have a width equal to about one-quarter of the wavelength of the acoustic standing wave generated in the flow chamber 110 of the device. In other embodiments, the slots 165 in the particulate screen 160 can have a width of between about 0.005 inches and 0.02 inches and a length of between about 0.25 inches and 0.75 inches. Again, the slots 165 in the particulate screen 160 are aligned with the planes of concentrated particles in the flow chamber 110.

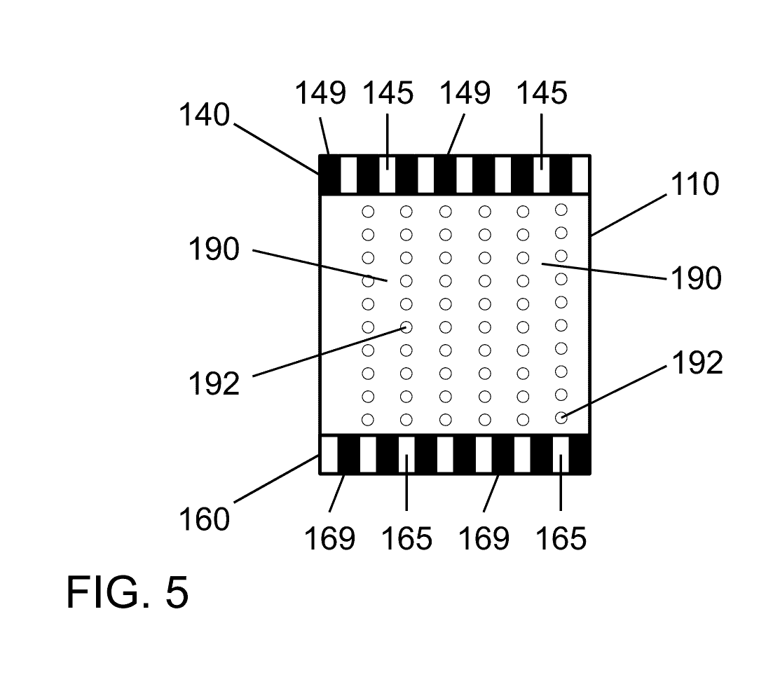

As explained herein, notwithstanding their substantially identical structure, the particulate screen 140 and the fluid screen 160 have opposite functions. More particularly, the slots 145 in the particulate screen 140 are aligned with the flow chamber 110 so as to allow the passage of clarified fluid therethrough, while the slots 165 in the fluid screen 160 are aligned with the flow chamber 110 so as to allow the passage of particles or particulate matter therethrough. FIG. 5 is a cross-sectional view of the flow chamber 110, the particulate screen 140, and the fluid screen 160 of the device 100 of FIG. 1 and FIG. 2. FIG. 5 shows the arrangement of the particulate screen 140 and the fluid screen 160 with respect to the flow chamber 110. As seen in FIG. 5, the slots 145 in the particulate screen 140 are aligned with the areas of clarified fluid 190 in the flow chamber 110, and the slots 165 in the fluid screen 160 are aligned with the areas of concentrated particles 192 in the flow chamber 110. Likewise, the bars 149 of the particulate screen 140 are aligned with the areas of concentrated particles 192 in the flow chamber 110, and the bars 169 of the fluid screen 160 are aligned with the areas of clarified fluid 190 in the flow chamber 110. In this way, the slots 145 in the particulate screen 140 are offset from (i.e., unaligned with) the slots 165 in the fluid screen 160, and the bars 149 of the particulate screen 140 are offset from (i.e., unaligned with) the bars 169 of the fluid screen 160, as shown in FIG. 5. As a result, clarified fluid will flow through particulate screen 140 and particles will be retarded by the particulate screen 140, while particles will pass through the fluid screen 160 and the clarified fluid layers will be retarded by the fluid screen 160.

With reference again to FIG. 1, the device 100 may further include an alignment screen between the incoming fluid flow and the flow chamber. In this embodiment, the device 100 includes two such alignment screens 150. One alignment screen 150 is located on wall 124 between side opening 116 and the flow chamber 110, and another alignment screen 150 is located on wall 126 between side opening 117 and the flow chamber 110. Wall 124 is located opposite wall 126 (i.e., on an opposite side of the flow chamber 110), such that the alignment screens 150 are located opposite one another.

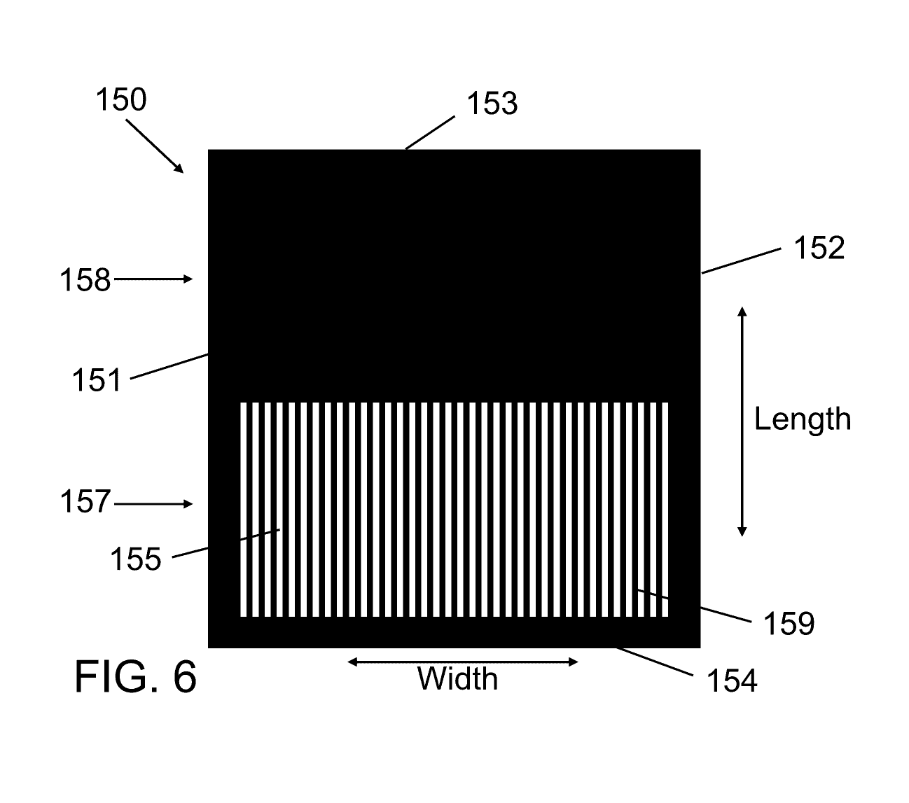

A plan view of one exemplary embodiment of the alignment screen 150 is shown in FIG. 6. The alignment screen has a first side 151, a second side 152 opposite the first side, a third side 153 between the first side and the second side, and a fourth side 154 opposite the third side and also located between the first side and the second side. The first side 151 and the second side 152 define a width, and the third side 153 and the fourth side define a length, of the alignment screen. These four sides define a perimeter of the alignment screen. The exact shape of the perimeter is not significant.

The alignment screen 150 includes a plurality of slots 155 therein, and a plurality of bars 159. Each slot is surrounded by two bars, and each bar is surrounded by two slots. In the embodiment illustrated, the slots 155 are longitudinal slots running the width of the alignment screen 150 and about half the length of the alignment screen 150. Put another way, the slots 155 in the alignment screen 150 run widthwise from one side thereof to the other (i.e., from first side 151 to second side 152) and span about half the width of the alignment screen 150 from the top to the bottom (i.e., about half the width from the third side 153 to the fourth side 154). In particular embodiments, the slots 155 are arranged in a single row 157 and the rest of the inlet screen 150 is a solid plate portion 158 without slots therein. The solid plate portion 158 of the alignment screen 150 increases the structural integrity of the alignment screen 150.

While the embodiment of the alignment screen 150 depicted in FIG. 6 shows the solid plate portion 158 comprising about half the height of the inlet screen 150, with the slotted portion 157 comprising the remaining half, it is to be understood that the alignment screen 150 can be designed with the slotted or solid portions comprising more or less than half of the alignment screen 150. For example, in certain embodiments, the slots 155 in the alignment screen 150 have a length of between about 0.25 inches and 0.75 inches (for an alignment screen length of 1″ total). The slots 155 in the alignment screen 150 can also have a width of between about 0.005 inches and 0.02 inches. Put another way, the slots 155 in the alignment screen 150 can comprise about half of the length of the inlet screen 150.

In the operating method described in FIG. 2, fluid flows into the flow chamber 110 through the side openings 116, 117. As illustrated in FIG. 1, the side openings 116, 117 are in the form of a plenum/chamber. The plenum volume provides flow diffusion and dramatically reduces incoming flow non-uniformities. Generally speaking, the mixture of host fluid/particulate flows in through an upper end of the plenum. The mixture fills up the plenum and then flows horizontally into the flow chamber through the alignment screen 150, which has slots located in the lower end of the plenum. This action reduces/eliminates flow pulsations and flow non-uniformities that result from pumps, hosing and horizontal inlet flow where gravity effects dominate. In these embodiments where the mixture flows into the flow chamber through the side openings 116, 117, the shape of the slots is not significant. They can take the form of a single slot, or lines of holes. The operation of the plenum/alignment screen is very similar to the dump diffuser described with reference to FIGS. 17-19 of U.S. patent application Ser. No. 14/791,115, filed Jul. 2, 2015, which is hereby fully incorporated by reference. In this operating method depicted in FIG. 2, the alignment screens 150 are downstream of the side openings 116 and 117, and the alignment screens 150 are upstream of the flow chamber 110.

As explained above, the particulate outlet 112 generally allows for egress or collection of particles, cells, or the like from the flow chamber 110. In comparison, the first opening 114 and side openings 116 and 117 generally allow for fluid ingress or egress from the flow chamber 110, as desired for a particular application. For example, as explained above, in the embodiment of the device 100 shown in FIG. 2, the side openings 116 and 117 can be configured to operate as inlets, such that the host fluid/particle mixture is flowed into the flow chamber 110 via side openings 116 and 117. In FIG. 2, the first opening 114 operates as an outlet for clarified fluid to exit the flow chamber 110.

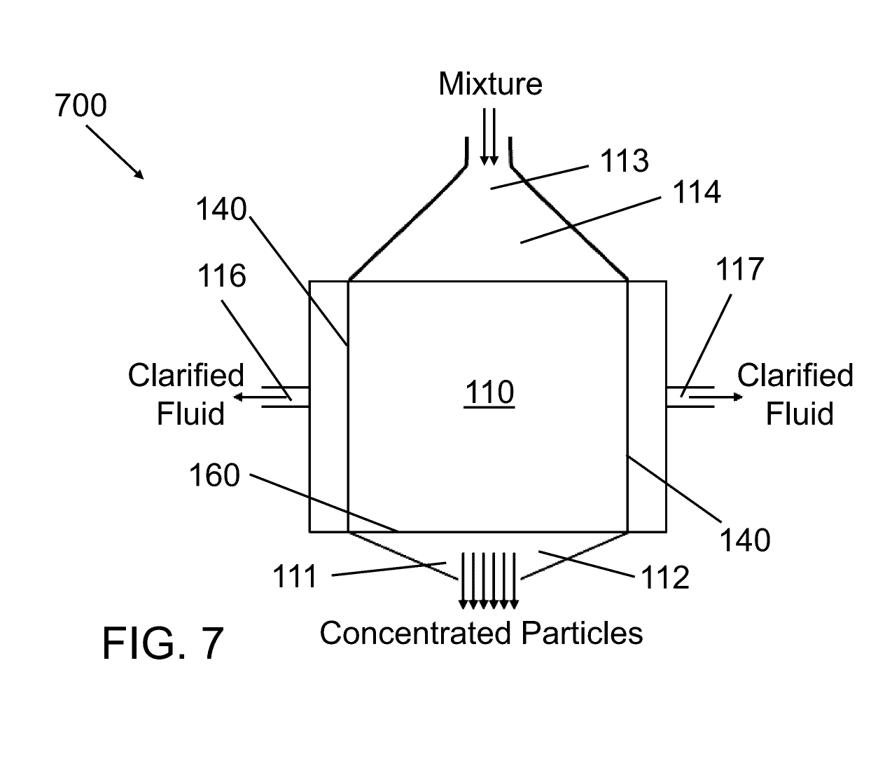

It is to be understood that the first opening 114 and the side openings 116 and 117 can be configured to operate as either inlets or outlets for the device, as desired. For example, in a second exemplary acoustophoretic device 700 depicted in FIG. 7, the mixture of the host fluid and the second fluid or particulate enters the device at the second end 113 (i.e., the first opening is at the top end of the device). In other words, in the exemplary acoustophoretic device 700 depicted in FIG. 7, the first opening 114 is generally configured to operate as an inlet for the mixture of the host fluid and the second fluid or particulate, the side openings 116 and 117 are generally configured to operate as outlets for the clarified fluid, and the particulate outlet 112 is located at the first end 111 of the device 700 (i.e., the particulate outlet is located at the bottom end of the device). The mixture then flows from the second end 113 of the device into the flow chamber 110. Upon separation of the second fluid or particulate from the host fluid by operation of the acoustic standing wave, the now-clarified host fluid passes through particulate screens 140 and out of the device via the side openings 116 and 117. Each particulate screen 140 operates as described above; only the location of the screen has changed, since the location where the clarified fluid is exiting has changed. Generally speaking, the particulate screen is always upstream of the clarified fluid outlet. Again, the slots in the particulate screen 140 are aligned so as to permit the passage of clarified fluid running between the planes of particles therethrough, while preventing or retarding the passage of particles or particulate matter therethrough. The concentrated particle planes, on the other hand, can pass through fluid screen 160 and be collected or removed from the device via particulate outlet 112. The side openings 116 and 117 can operate in this mode without the use of a plenum construction on either of the side openings.

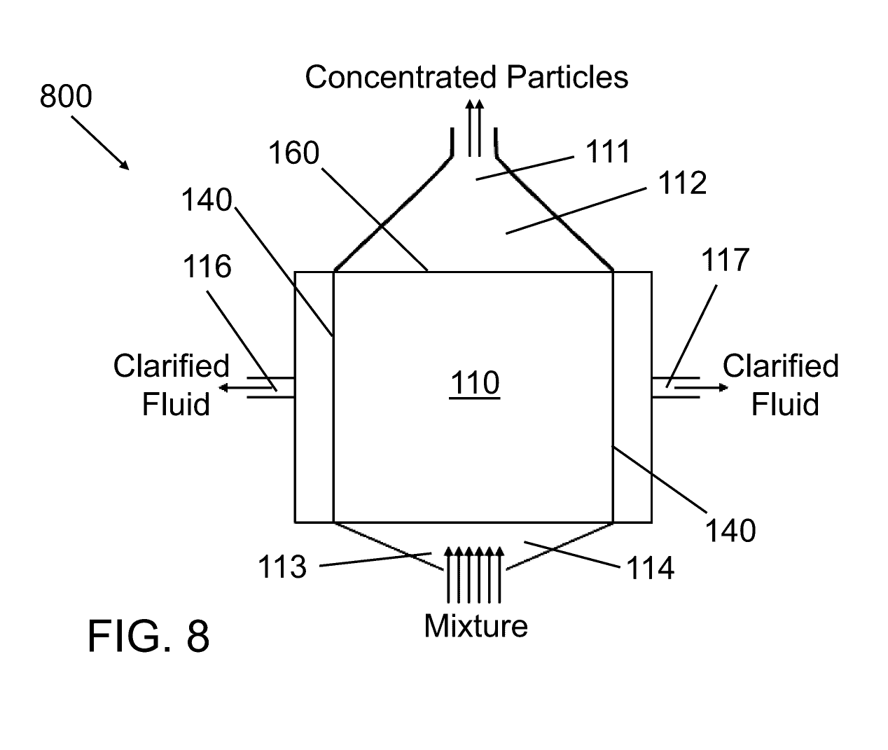

FIG. 8 illustrates a third exemplary operating method for a device 800. Here, the mixture of the host fluid and the second fluid or particulate is one where the second fluid or particular is less dense than the host fluid. One example would be where the host fluid is water, and the particulate is oil droplets. Here, the mixture enters the device at the second end 113 (i.e., the first opening is at the bottom end of the device). In other words, the first opening 114 is configured to operate as an inlet for the mixture of the host fluid and the second fluid or particulate, the side openings 116 and 117 are generally configured to operate as outlets for the clarified fluid, and the particulate outlet 112 is located at the first end 111 of the device 800 (i.e., the particulate outlet is now at the top end of the device). The mixture then flows from the second end 113 of the device into the flow chamber 110. Upon separation of the second fluid or particulate from the host fluid by operation of the acoustic standing wave, the now-clarified host fluid can pass through particulate screens 140 and out of the device via the side openings 116 and 117. The concentrated particle planes, on the other hand, now flow upwards through fluid screen 160 and are collected or removed from the device via particulate outlet 112.

As explained in detail above, the particulate screen(s) and fluid screen(s) of the presently disclosed devices and methods generally pre-condition and/or post-condition the host fluid containing particles. The particulate screen and fluid screen(s) located downstream of the acoustic standing wave generated by the ultrasonic transducer and reflector selectively permit the passage of one or the other separated layers of particles/fluids therethrough. Put another way, the slots in the particulate screen are aligned to match up with the areas of clarified fluid created in the acoustic standing wave. As a result, the clarified fluid can flow out of the flow chamber while the particulate remains retained in the flow chamber by the particulate screen. Likewise, the slots in the fluid screen are aligned to match up with the areas of concentrated particles created in the acoustic standing wave. As a result, the concentrated particles can be collected or removed from the flow chamber via the particulate outlet while the streams of clarified fluid are prevented or retarded from exiting through the particulate outlet by the fluid screen. In this regard, it is important that the slots in the particulate and fluid screens are sized and located so as to be aligned with the frequency of the acoustic standing wave generated by the transducer and reflector. As a result thereof, the areas of clarified fluid align with the slots in the particulate screen (with the areas of concentrated particles aligned with the bars of the particulate screen), and the areas of concentrated align with the slots in the fluid screen (with the areas of clarified fluid aligned with the bars of the fluid screen),

As previously explained, the ultrasonic transducer and reflector are located on opposite sides of the flow chamber. In this way, one or more acoustic standing waves are created between the ultrasonic transducer and reflector.

Prior to discussing further optimization of the systems, it is helpful to provide an explanation now of how multi-dimensional acoustic standing waves are generated. The multi-dimensional acoustic standing wave needed for particle collection is obtained by driving an ultrasonic transducer at a frequency that both generates the acoustic standing wave and excites a fundamental 3D vibration mode of the transducer crystal. Perturbation of the piezoelectric crystal in an ultrasonic transducer in a multimode fashion allows for generation of a multidimensional acoustic standing wave. A piezoelectric crystal can be specifically designed to deform in a multimode fashion at designed frequencies, allowing for generation of a multi-dimensional acoustic standing wave. The multi-dimensional acoustic standing wave may be generated by distinct modes of the piezoelectric crystal such as a 3×3 mode that would generate multidimensional acoustic standing waves. A multitude of multidimensional acoustic standing waves may also be generated by allowing the piezoelectric crystal to vibrate through many different mode shapes. Thus, the crystal would excite multiple modes such as a 0×0 mode (i.e. a piston mode) to a 1×1, 2×2, 1×3, 3×1, 3×3, and other higher order modes and then cycle back through the lower modes of the crystal (not necessarily in straight order). This switching or dithering of the crystal between modes allows for various multidimensional wave shapes, along with a single piston mode shape to be generated over a designated time.

The scattering of the acoustic field off the particles results in a three dimensional acoustic radiation force, which acts as a three-dimensional trapping field. The acoustic radiation force is proportional to the particle volume (e.g. the cube of the radius) when the particle is small relative to the wavelength. It is proportional to frequency and the acoustic contrast factor. It also scales with acoustic energy (e.g. the square of the acoustic pressure amplitude). When the acoustic radiation force exerted on the particles is stronger than the combined effect of fluid drag force and buoyancy and gravitational force, the particles are trapped within the acoustic standing wave field. This results in concentration, agglomeration and/or coalescence of the trapped particles. Relatively large solids of one material can thus be separated from smaller particles of a different material, the same material, and/or the host fluid through enhanced gravitational separation.

The multi-dimensional standing wave generates acoustic radiation forces in both the axial direction (i.e., in the direction of the standing wave, between the transducer and the reflector, perpendicular to the flow direction) and the lateral direction (i.e., in the flow direction). As the mixture flows through the acoustic chamber, particles in suspension experience a strong axial force component in the direction of the standing wave. Since this acoustic force is perpendicular to the flow direction and the drag force, it quickly moves the particles to pressure nodal planes or anti-nodal planes, depending on the contrast factor of the particle. The lateral acoustic radiation force then acts to move the concentrated particles towards the center of each planar node, resulting in agglomeration or clumping. The lateral acoustic radiation force component has to overcome fluid drag for such clumps of particles to continually grow and then drop out of the mixture due to gravity. Therefore, both the drop in drag per particle as the particle cluster increases in size, as well as the drop in acoustic radiation force per particle as the particle cluster grows in size, must be considered for the acoustic separator device to work effectively. In the present disclosure, the lateral force component and the axial force component of the multi-dimensional acoustic standing wave are of the same order of magnitude. In some particular embodiments, the ratio of the lateral force component to the axial force component is about 0.5 or less. In this regard, it is noted that in a multi-dimensional acoustic standing wave, the axial force is stronger than the lateral force, but the lateral force of a multi-dimensional acoustic standing wave is much higher than the lateral force of a planar standing wave, usually by two orders of magnitude or more.

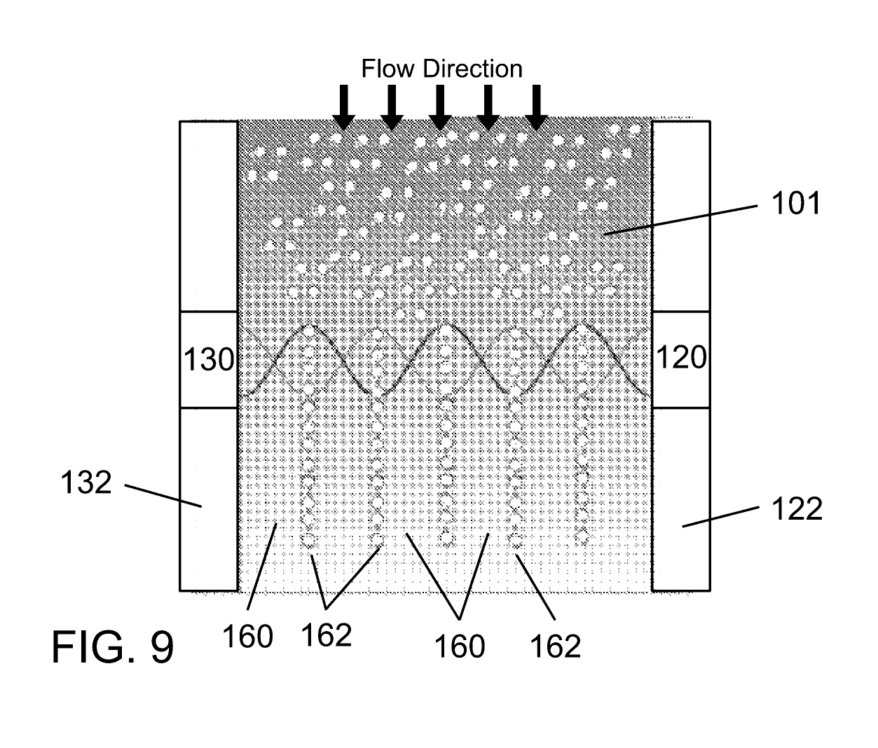

FIG. 9 illustrates one exemplary arrangement of the system, which includes a transducer 120 and reflector 130. Generally, the transducer 120 includes a piezoelectric material driven by a voltage signal to create an acoustic standing wave in the flow chamber. The incoming fluid mixture 101 is a mixture of host fluid and particles. The transducer can be driven so as to cause the particles to collect, agglomerate, aggregate, clump, or coalesce at the nodes or anti-nodes of the acoustic standing wave, depending on the particles' or secondary fluid's acoustic contrast factor relative to the host fluid. This causes the fluid mixture 101 to be separated into layers 160 of clarified fluid that have a lower concentration of particles, and into layers or planes 162 in which the particle concentration is enhanced (or more generally the concentration of dispersed species is higher, or more rich). This results in a stratified flow, with alternating layers of clarified fluid and particle-rich fluid. It is noted that the acoustic standing wave is driven at a power sufficient to cause alignment of the particles within planes, but is not necessarily driven such that the particles are held within the acoustic standing wave until the particles fall out due to gravity or buoyancy. It is contemplated that the fluid drag force may be great enough to keep particles flowing out of the acoustic standing wave. The particles flows through the slots in the outlet screen, and can then be collected via the particulate outlet. The clarified fluid flows through the slots of the inlet screen and can be recovered via the clarified fluid outlet. Further downstream processing can occur, depending on whether it is desired to collect the particles or some other material still present within the host fluid. For example, the particles could be cells from a bioreactor, such as Chinese hamster ovary (CHO) cells, NS0 hybridoma cells, baby hamster kidney (BHK) cells, or human cells. In such a situation, it may be desired to purify and obtain biomolecules such as recombinant proteins or monoclonal antibodies produced by such cells and remaining in the clarified fluid exiting through the inlet screens. The clarified fluid can be subjected to further downstream processing and filtration to obtain the biomolecules. Alternatively, the particles themselves may be the desired product, in which case the volume of liquid that must go through further downstream processing has been reduced.

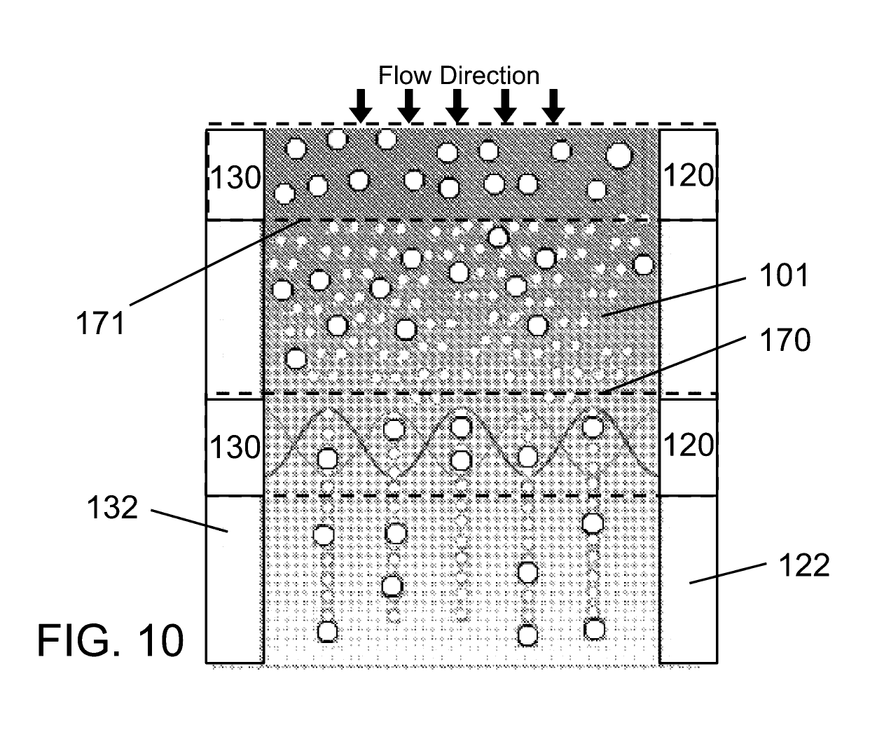

Turning now to FIG. 10, another exemplary arrangement of the transducer 120 and reflector 130 is illustrated. In this arrangement, the transducer 120 and reflector 130 define a primary transducer-reflector pair 170. The primary transducer-reflector pair 170 is generally operated to cause trapping and agglomeration of a second fluid or particulate in the acoustic standing wave, as explained in detail herein. A secondary transducer-reflector pair 171 is also depicted in FIG. 10 upstream of the primary transducer-reflector pair 170. The secondary transducer-reflector pair 171 is generally operated so as to cause cavitation, resulting in micro-bubbles in the host fluid/particle mixture 101. The micro-bubbles assist in flocculation or aggregation of the second fluid or particulate by the primary transducer-reflector pair 170. Put another way, the secondary transducer-reflector pair 171 causes cavitation in the flow chamber upstream of the primary transducer-reflector pair 170, creating micro-bubbles. Attachment to the bubbles allows for easier separation of the second fluid or particulate from the host fluid using the primary transducer-reflector pair 170, which aligns the second fluid or particulate into planes in the flow chamber.

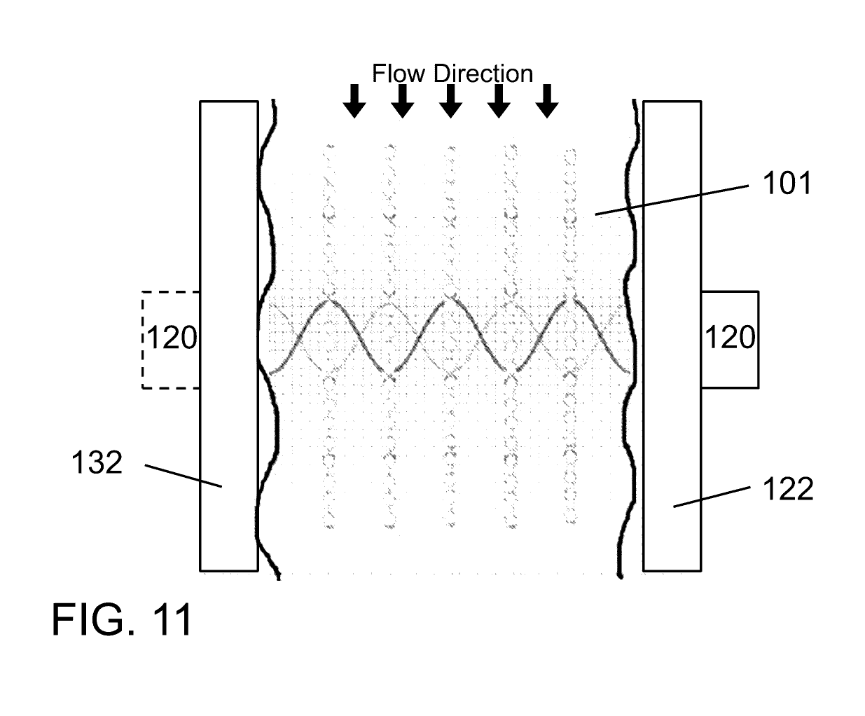

Yet another exemplary arrangement of the transducer 120 and reflector 130 is illustrated in FIG. 11. In this arrangement, the transducer(s) 120 can be driven to set the entire system into vibration at the resonant frequency of wall 122/132. By exciting wall 122/132, acoustic standing waves can be created in the flow chamber, which can be used to separate the mixture 101 into planes of particles and layers of clarified fluid therein. As illustrated here, one transducer 120 is present on the exterior (or backside) of wall 122. Optional transducer 120 is illustrated in dashed line on the exterior of wall 132. When only one transducer is used, the transducer causes the wall the vibrate, which will reflect off of the opposite wall.

Some further explanation of the ultrasonic transducers used in the devices, systems, and methods of the present disclosure may be helpful as well. In this regard, the transducers use a piezoelectric crystal, usually made of PZT-8 (lead zirconate titanate). Such crystals may have a 1 inch diameter and a nominal 2 MHz resonance frequency, and may also be of a larger size. Each ultrasonic transducer module can have only one crystal, or can have multiple crystals that each act as a separate ultrasonic transducer and are either controlled by one or multiple amplifiers. The crystals can be square, rectangular, irregular polygon, or generally of any arbitrary shape. The transducer(s) is/are used to create a pressure field that generates forces of the same order of magnitude both orthogonal to the standing wave direction (lateral) and in the standing wave direction (axial).

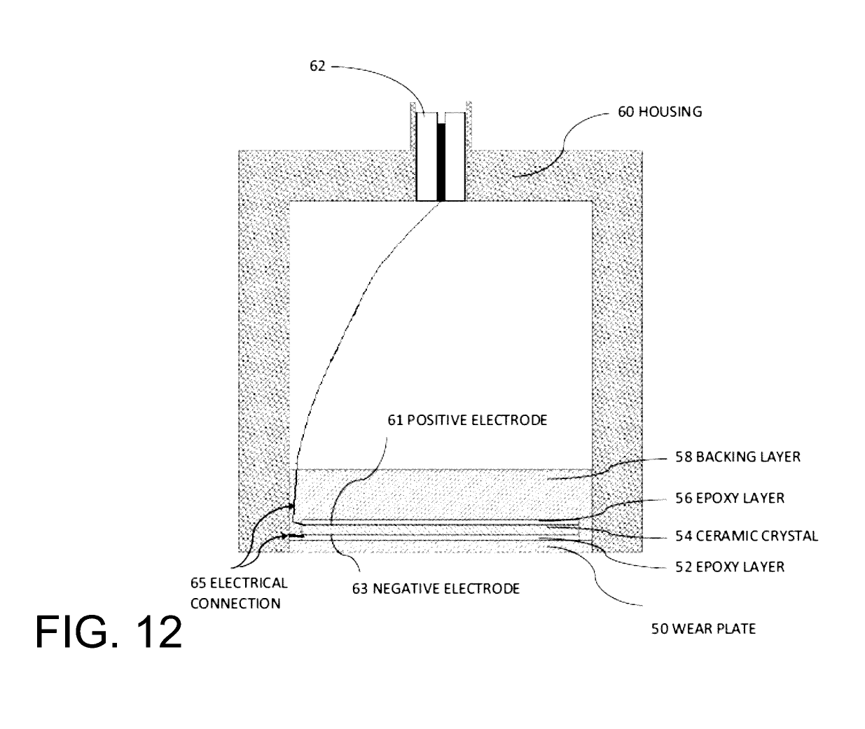

FIG. 12 is a cross-sectional diagram of a conventional ultrasonic transducer. This transducer has a wear plate 50 at a bottom end, epoxy layer 52, ceramic crystal 54 (made of, e.g. PZT), an epoxy layer 56, and a backing layer 58. On either side of the ceramic crystal, there is an electrode: a positive electrode 61 and a negative electrode 63. The epoxy layer 56 attaches backing layer 58 to the crystal 54. The entire assembly is contained in a housing 60 which may be made out of, for example, aluminum. An electrical adapter 62 provides connection for wires to pass through the housing and connect to leads (not shown) which attach to the crystal 54. Typically, backing layers are designed to add damping and to create a broadband transducer with uniform displacement across a wide range of frequency and are designed to suppress excitation at particular vibrational eigen-modes. Wear plates are usually designed as impedance transformers to better match the characteristic impedance of the medium into which the transducer radiates.

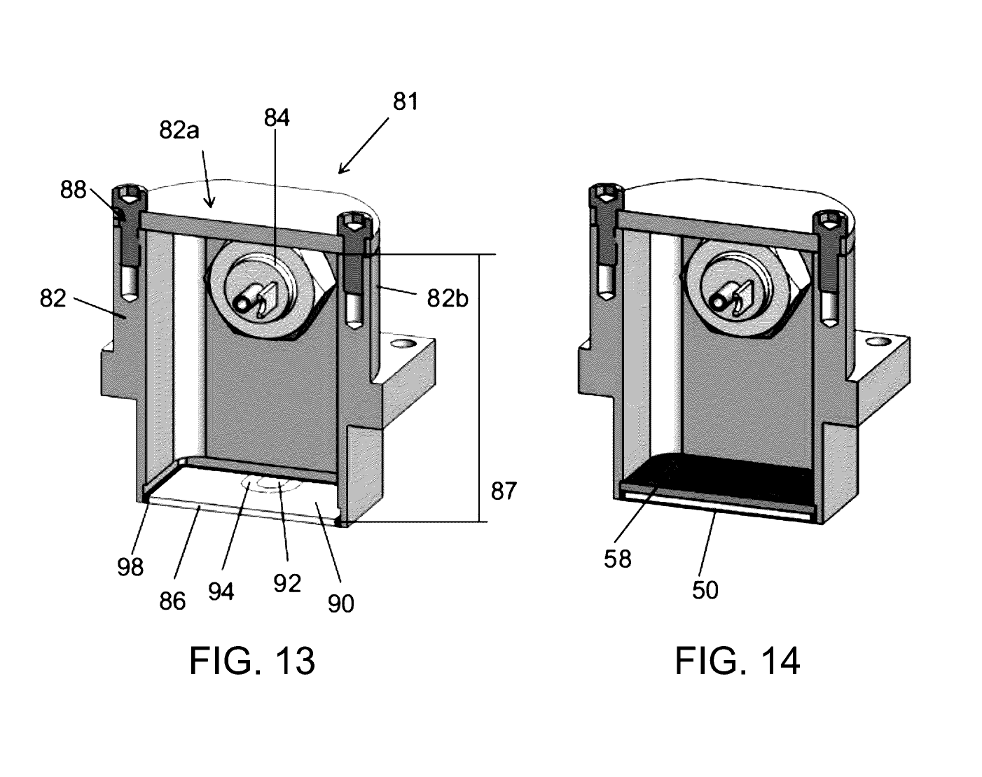

FIG. 13 is a cross-sectional view of an ultrasonic transducer 81 of the present disclosure. Transducer 81 is shaped as a disc or a plate, and has an aluminum housing 82. The piezoelectric crystal is a mass of perovskite ceramic crystals, each consisting of a small, tetravalent metal ion, usually titanium or zirconium, in a lattice of larger, divalent metal ions, usually lead or barium, and O2— ions. As an example, a PZT (lead zirconate titanate) crystal 86 defines the bottom end of the transducer, and is exposed from the exterior of the housing. The crystal is supported on its perimeter by a small elastic layer 98, e.g. silicone or similar material, located between the crystal and the housing. Put another way, no wear layer is present. In particular embodiments, the crystal is an irregular polygon, and in further embodiments is an asymmetrical irregular polygon.

Screws 88 attach an aluminum top plate 82a of the housing to the body 82b of the housing via threads. The top plate includes a connector 84 for powering the transducer. The top surface of the PZT crystal 86 is connected to a positive electrode 90 and a negative electrode 92, which are separated by an insulating material 94. The electrodes can be made from any conductive material, such as silver or nickel. Electrical power is provided to the PZT crystal 86 through the electrodes on the crystal. Note that the crystal 86 has no backing layer or epoxy layer. Put another way, there is an air gap 87 in the transducer between aluminum top plate 82a and the crystal 86 (i.e. the air gap is completely empty). A minimal backing 58 and/or wear plate 50 may be provided in some embodiments, as seen in FIG. 14.

The transducer design can affect performance of the system. A typical transducer is a layered structure with the ceramic crystal bonded to a backing layer and a wear plate. Because the transducer is loaded with the high mechanical impedance presented by the standing wave, the traditional design guidelines for wear plates, e.g., half wavelength thickness for standing wave applications or quarter wavelength thickness for radiation applications, and manufacturing methods may not be appropriate. Rather, in one embodiment of the present disclosure the transducers, there is no wear plate or backing, allowing the crystal to vibrate in one of its eigenmodes (i.e. near eigenfrequency) with a high Q-factor. The vibrating ceramic crystal/disk is directly exposed to the fluid flowing through the flow chamber.

Removing the backing (e.g. making the crystal air backed) also permits the ceramic crystal to vibrate at higher order modes of vibration with little damping (e.g. higher order modal displacement). In a transducer having a crystal with a backing, the crystal vibrates with a more uniform displacement, like a piston. Removing the backing allows the crystal to vibrate in a non-uniform displacement mode. The higher order the mode shape of the crystal, the more nodal lines the crystal has. The higher order modal displacement of the crystal creates more trapping lines, although the correlation of trapping line to node is not necessarily one to one, and driving the crystal at a higher frequency will not necessarily produce more trapping lines.

In some embodiments, the crystal may have a backing that minimally affects the Q-factor of the crystal (e.g. less than 5%). The backing may be made of a substantially acoustically transparent material such as balsa wood, foam, or cork which allows the crystal to vibrate in a higher order mode shape and maintains a high Q-factor while still providing some mechanical support for the crystal. The backing layer may be a solid, or may be a lattice having holes through the layer, such that the lattice follows the nodes of the vibrating crystal in a particular higher order vibration mode, providing support at node locations while allowing the rest of the crystal to vibrate freely. The goal of the lattice work or acoustically transparent material is to provide support without lowering the Q-factor of the crystal or interfering with the excitation of a particular mode shape.

Placing the crystal in direct contact with the fluid also contributes to the high Q-factor by avoiding the dampening and energy absorption effects of the epoxy layer and the wear plate. Other embodiments may have wear plates or a wear surface to prevent the PZT, which contains lead, contacting the host fluid. This may be desirable in, for example, biological applications such as separating blood. Such applications might use a wear layer such as chrome, electrolytic nickel, or electroless nickel. Chemical vapor deposition could also be used to apply a layer of poly(p-xylylene) (e.g. Parylene) or other polymers or polymer films. Organic and biocompatible coatings such as silicone or polyurethane are also usable as a wear surface.

The acoustophoretic devices and methods described herein are useful for pre-conditioning a second fluid or particulate within a host fluid by aligning the second fluid or particulate into planes, which advantageously allows for easier separation of the second fluid or particulate from the host fluid. In this regard, the second fluid or particulate may be subsequently separated from the host fluid by any known filtration or processing, such as by collecting the second fluid or particulate from the particulate outlet and feeding the same to another filtration process.

The present disclosure has been described with reference to exemplary embodiments. Obviously, modifications and alterations will occur to others upon reading and understanding the preceding detailed description. It is intended that the present disclosure be construed as including all such modifications and alterations insofar as they come within the scope of the appended claims or the equivalents thereof.

1. An acoustophoretic device, comprising:
a flow chamber having a particulate outlet at a first end of the flow chamber and a first opening at a second end of the flow chamber opposite the first end thereof;
at least one ultrasonic transducer located on a wall of the flow chamber, the at least one ultrasonic transducer including a piezoelectric material driven by a voltage signal to create an acoustic standing wave in the flow chamber;
a reflector located on a wall on the opposite side of the flow chamber from the at least one ultrasonic transducer;
at least one side opening located on a wall of the flow chamber between the reflector and the at least one ultrasonic transducer;
a fluid screen located between the particulate outlet and the flow chamber, the fluid screen including a plurality of slots therein.

2. The acoustophoretic device of claim 1, wherein the slots in the outlet screen have a width equal to about one-quarter of the wavelength of the acoustic standing wave.

3. The acoustophoretic device of claim 1, wherein the slots in the outlet screen have a width of between about 0.005 inches and 0.02 inches and a height of between about 0.25 inches and 0.75 inches.

4. The acoustophoretic device of claim 1, further comprising at least one particulate screen located between the flow chamber and either (i) the first opening or (ii) the at least one side opening, the particulate screen including a plurality of slots therein.

5. The acoustophoretic device of claim 4, wherein the slots in the at least one particulate screen have a width of between about 0.005 inches and 0.02 inches and a height of between about 0.25 inches and about 0.75 inches.

6. The acoustophoretic device of claim 4, wherein the slots of the fluid screen are offset from the slots of the at least one particulate screen.

7. The acoustophoretic device of claim 1, further comprising an alignment screen located between the at least one side opening and the flow chamber.

8. The acoustophoretic device of claim 1, wherein the acoustic standing wave is a multi-dimensional acoustic standing wave.

9. A method for conditioning a second fluid or a particulate within a host fluid, comprising:
flowing a mixture of the host fluid and the second fluid or particulate through an acoustophoretic device, the acoustophoretic device comprising:
a flow chamber having a particulate outlet at a first end of the flow chamber and a first opening at a second end of the flow chamber opposite the first end thereof;
at least one ultrasonic transducer located on a wall of the flow chamber, the at least one ultrasonic transducer including a piezoelectric material driven by a voltage signal to create an acoustic standing wave in the flow chamber;
a reflector located on a wall on the opposite side of the flow chamber from the at least one ultrasonic transducer;
at least one side opening located on a wall of the flow chamber between the reflector and the at least one ultrasonic transducer; and
a fluid screen located between the particulate outlet and the flow chamber, the fluid screen including a plurality of slots therein;
sending a voltage signal to drive the at least one ultrasonic transducer to create the acoustic standing wave in the flow chamber to create aligned and separated layers of (i) the host fluid and (ii) the second fluid or particulate; and
using the fluid screen to separate the layers of the second fluid or particulate from the layers of the host fluid.

10. The method of claim 9, wherein the slots in the fluid screen have a width equal to about one-quarter of the wavelength of the multi-dimensional standing wave.

11. The method of claim 9, wherein the at least one ultrasonic transducer excites the wall of the flow chamber to create the acoustic standing wave.

12. The method of claim 9, wherein the acoustophoretic device further comprises at least one particulate screen located between the flow chamber and either (i) the first opening or (ii) the at least one side opening, the particulate screen including a plurality of slots therein.

13. The method of claim 12, wherein the slots of the fluid screen are aligned with the separated layers of the second fluid or particulate, and the slots of the at least one particulate screen are aligned with the layers of the host fluid.

14. The method of claim 12, wherein the slots in the at least one particulate screen have a width of between about 0.005 inches and 0.02 inches and a height of between about 0.25 inches and about 0.75 inches.

15. The method of claim 9, wherein the acoustic standing wave is a multi-dimensional acoustic standing wave.

16. The method of claim 9, wherein the at least one transducer and the reflector define a primary transducer-reflector pair, and the acoustophoresis device further comprises a secondary transducer-reflector pair located upstream of the primary transducer-reflector pair, the secondary transducer-reflector causing cavitation resulting in micro-bubbles in the host fluid that assist in flocculation or aggregation of the second fluid or particulate prior to separation into layers by the primary transducer-reflector pair.

17. The method of claim 9, wherein the slots in the fluid screen are arranged in two rows of longitudinal slots separated by a divider running therebetween.

18. The method of claim 9, wherein the mixture flows into the flow chamber through the first opening, and the separated layers of the host fluid exit the flow chamber through the at least one side opening.

19. The method of claim 9, wherein the mixture flows into the flow chamber through the at least one side opening, and the separated layers of the host fluid exit the flow chamber through the first opening.

20. A method for separating a second fluid or a particulate from a host fluid, comprising:
flowing a mixture of the host fluid and the second fluid or particulate into a flow chamber;
generating an acoustic standing wave in the flow chamber to create aligned and separated layers of (i) the host fluid and (ii) the second fluid or particulate; and
using a fluid screen to separate the layers of the second fluid or particulate from the layers of the host fluid.
